(19) United States
(12) Patent Application Publication
(10) Pub. No.: US 2026/0248637 A1
(43) Pub. Date: Aug. 27, 2026

(54) FLUID COLLECTION DEVICES, SYSTEMS, AND METHODS

(71) Applicant: PureWick Corporation, Covington, GA (US)

(72) Inventors: Ashley Marie Johannes, Atlanta, GA (US); Hollie Trullenque, Houston, TX (US); Megan Evans, Grove City, OH (US); Michelle Root, Malvern, PA (US)

(21) Appl. No.: 19/535,649

(22) Filed: Feb. 10, 2026

Related U.S. Application Data
(63) Continuation of application No. 17/625,941, filed on Jul. 8, 2020, now Pat. No. 12,569,364.
(60) Provisional application No. 62/873,048, filed on Jul. 11, 2019.

(51) Int. Cl. A61F 5/451; A61L 31/02; A61L 31/14; A61L 31/16

(52) U.S. Cl. CPC A61F 5/451; A61L 31/022; A61L 31/146; A61L 31/16; A61L 2300/406

(57) ABSTRACT

Examples relate to systems, devices, and methods for removing fluid from a fluid collection device using a vacuum source operably coupled thereto. The fluid collection devices include urine collection devices shaped to complement the female or male anatomy near the respective urethras and the vacuum source is operably coupled to the fluid collection device via one or more sections of conduit. The fluid collection devices include antimicrobial materials in or on one or more components thereof.

### CROSS REFERENCE TO RELATED APPLICATIONS

This application is a continuation of U.S. application Ser. No. 17/625,941 filed on 10 Jan. 2022, which is a U.S. National Stage of International Application No. PCT/US2020/041249 filed on 8 Jul. 2020, which claims priority to U.S. Patent Application No. 62/873,048 filed on 11 Jul. 2019, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to devices, systems, and methods of using fluid collection devices. In an embodiment, a fluid collection device is disclosed. The fluid collection device includes a fluid impermeable barrier having an outer surface and an inner surface, the inner surface at least partially defining a chamber, the fluid impermeable barrier also defining an opening extending therethrough, the opening configured to be positioned adjacent to a female urethra or have a male urethra positioned therethrough. The fluid collection device includes a wicking material disposed in the chamber. The fluid collection device includes an antimicrobial material carried by one or more of the fluid impermeable barrier or the wicking material.

In an embodiment, a fluid collection system is disclosed. The fluid collection system includes a fluid storage container configured to hold a fluid. The fluid collection system includes a fluid collection device fluidly coupled to the fluid storage container. The fluid collection device includes a fluid impermeable barrier having an outer surface and an inner surface, the inner surface at least partially defining a chamber, the fluid impermeable barrier also defining an opening configured to be positioned adjacent to a female urethra or have a male urethra positioned therethrough. The fluid collection device includes a wicking material disposed within the fluid impermeable barrier. The fluid collection device includes a conduit including an inlet and an outlet, the outlet being fluidly coupled to the fluid storage container and the inlet being positioned in a portion of the chamber selected to be at a gravimetrically low point of the fluid collection device when worn by a user. The fluid collection device includes an antimicrobial material carried by one or more of the fluid impermeable barrier or the wicking material. The fluid collection system includes a vacuum source fluidly coupled to one or more of the fluid storage container or the fluid collection device via the conduit, the vacuum source configured to draw fluid from the fluid collection device via the conduit.

In an embodiment, a method to collect fluid is disclosed. The method includes positioning an opening of a fluid collection device adjacent to a female urethra or around a male urethra. The fluid collection device of the method includes a fluid impermeable barrier having an outer surface and an inner surface, the inner surface at least partially defining a chamber, the fluid impermeable barrier also defining an opening configured to be positioned adjacent to a female urethra or have a male urethra positioned therethrough. The fluid collection device of the method includes a wicking material disposed within the fluid impermeable barrier. The fluid collection device of the method includes a conduit including an inlet and an outlet, the outlet being fluidly coupled to a fluid storage container and the inlet being positioned in a portion of the chamber selected to be at a gravimetrically low point of the fluid collection device when worn by a user. The fluid collection device of the method includes an antimicrobial material carried by one or more of the fluid impermeable barrier or the wicking material. The method includes receiving fluid from the female urethra or the male urethra into the chamber of the fluid collection device. The method includes applying suction with a vacuum source effective to suction the fluid from the chamber via a conduit disposed therein and fluidly coupled to the vacuum source.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device, according to an embodiment.

FIG. 1B is an exploded view of the fluid collection device of FIG. 1A.

FIG. 1C is a cross-sectional view of the fluid collection device of FIG. 1A taken along the plane A-A, according to an embodiment.

FIG. 2A is an isometric view of a fluid collection device, according to an embodiment.

FIG. 2B is a cross-sectional view of a fluid collection device of FIG. 2A along the plane B-B, according to an embodiment.

FIG. 3 is a block diagram of a system for fluid collection, according to an embodiment.

FIG. 4 is a flow diagram of a method to collect fluid, according to an embodiment.

### DETAILED DESCRIPTION

Embodiments disclosed herein are related to devices, systems, and methods of using fluid collection devices and systems. The devices, systems, and methods of using fluid collection devices and systems include an antimicrobial material therein or thereon to prevent infections, lower microbial growth rates, and control odors in the fluid collection device.

In some examples, a fluid collection device includes a fluid impermeable barrier that at least partially defines a chamber. The fluid impermeable barrier also defines an opening extending therethrough that is configured to be positioned adjacent to a female urethra or have a male urethra positioned therethrough. The fluid collection device includes a wicking material disposed within the chamber. The fluid collection device may also include a conduit disposed in the chamber. The fluid collection device includes an anti-microbial material carried by one or more of the fluid impermeable barrier, the wicking material, or the conduit. The anti-microbial material may be disposed in, on, or incorporated into any of the components of the fluid collection device.

The fluid collection devices disclosed herein are configured to collect fluid(s) from an individual. The fluid collected by the fluid collection devices may include urine. The fluid(s) collected by the fluid collection devices may include at least one of vaginal discharge, penile discharge, reproductive fluids, blood, sweat, or other bodily fluids. Accordingly, the fluids may present a source or nidus for harmful microbes, such as bacteria, fungi, viruses, or the like. By providing an antimicrobial material on one or more portions of the fluid impermeable barrier, the wicking material, or the conduit, the microbial (e.g., bacterial, fungal, etc.) growth is limited or stopped, thereby limiting or preventing the onset of infection and production of odors.

The fluid collection devices disclosed herein are configured to be used in fluid collection systems to collect and remove fluids from the wearer of the fluid collection device.

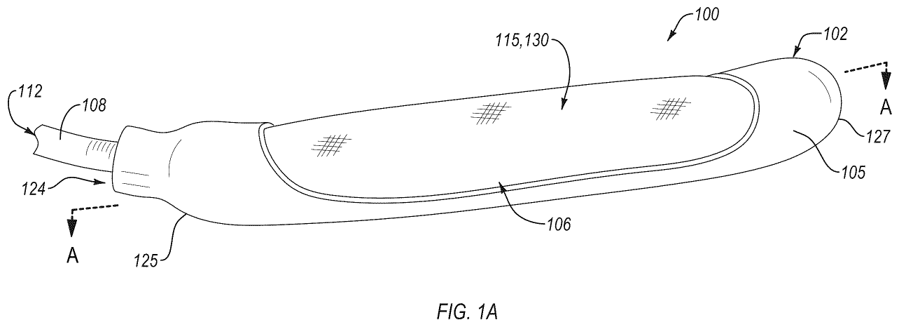

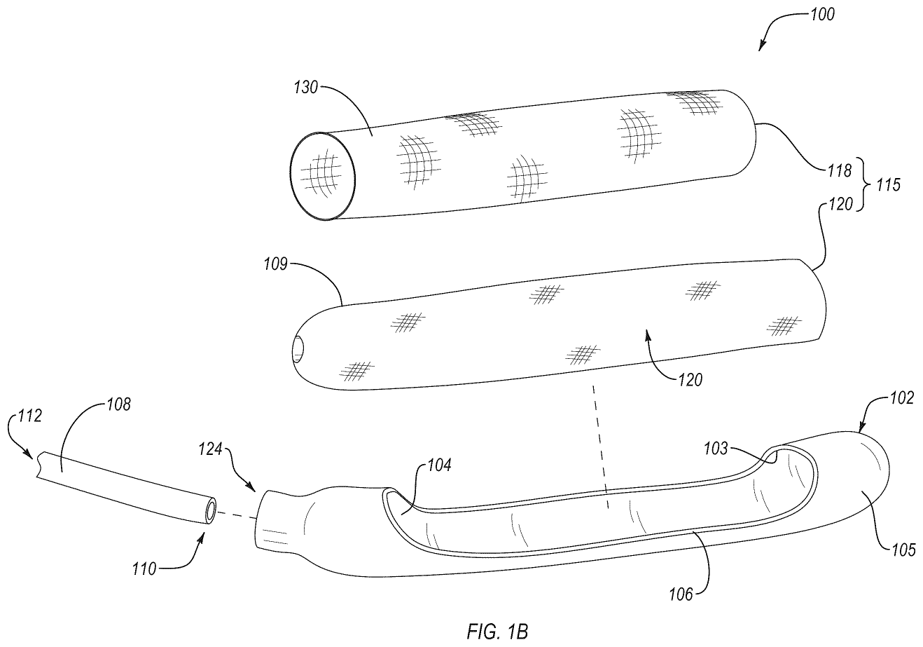

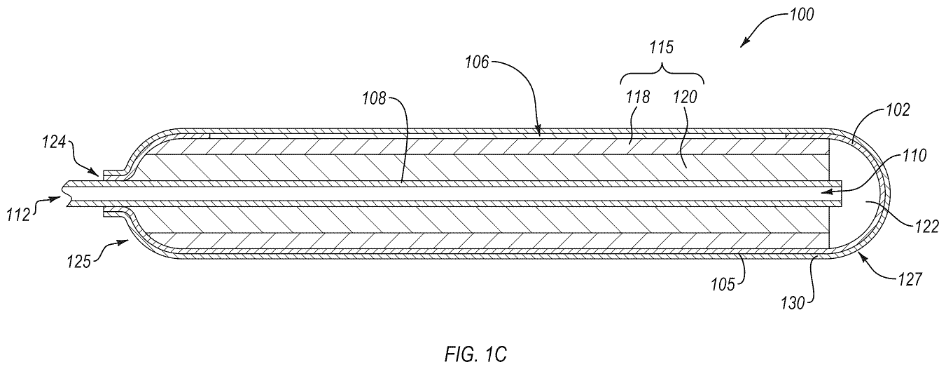

FIG. 1A is an isometric view of a fluid collection device 100, according to an embodiment. FIG. 1B is an exploded view of the fluid collection device 100 of FIG. 1A. The fluid collection device 100 is an example of a female fluid collection device for receiving and collecting fluid(s) from a female. The fluid collection device 100 includes a fluid impermeable barrier 102, wicking material 115 disposed in a chamber within the fluid impermeable barrier 102, an antimicrobial material 130, and an optional conduit 108 at least partially disposed within the chamber.

The fluid impermeable barrier 102 at least partially defines a chamber 104 (e.g., interior region) and an opening 106. For example, the interior surface(s) 103 of the fluid impermeable barrier 102 at least partially define the chamber 104 within the fluid collection device 100. The fluid impermeable barrier 102 at least temporarily retains the fluid(s) in the chamber 104. The fluid impermeable barrier 102 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 102 substantially prevents the fluid(s) from passing through the fluid impermeable barrier 102.

In some examples, the fluid impermeable barrier 102 may be tubular (ignoring the opening), such as substantially cylindrical (as shown), oblong, prismatic, or flattened tubes. The fluid impermeable barrier 102 may be sized to fit between the legs of a female user. During use, the outer surface 105 of the fluid impermeable barrier 102 may contact the wearer.

The opening 106 provides an ingress route for fluids to enter the chamber 104. The opening 106 may be defined by the fluid impermeable barrier 102, such as by an inner edge of the fluid impermeable barrier 102. For example, the opening 106 is formed in and extends through the fluid impermeable barrier 102, from the outer surface 105 to the inner surface 103, thereby enabling fluid(s) to enter the chamber 104 from outside of the fluid collection device 100. The opening 106 may be located and shaped to be positioned adjacent to a female urethra. At least a portion of wicking material(s) disposed in the chamber 104 may be exposed through the opening 106, to wick fluids inwardly into the chamber 104.

The fluid collection device 100 may be positioned proximate to the female urethra and urine may enter the chamber 104 via the opening 106. When in use, the opening 106 may be an elongated shape that extends from a first location below the urethral opening (e.g., at or near the anus or the vaginal opening) to a second location above the urethral opening (e.g., at or near the top of the vaginal opening or the pubic region).

The opening 106 may exhibit an elongated shape because the space between the legs of a female is relatively small when the legs of the female are closed, thereby only permitting the flow of the fluid(s) along a path that corresponds to the elongated shape of the opening 106 (e.g., longitudinally extending opening). The opening 106 in the fluid impermeable barrier 102 may exhibit a length that is measured along the longitudinal axis of the fluid collection device 100 that may be at least about 10% of the length of the fluid collection device 100, such as about 25% to about 50%, about 40% to about 60%, about 50% to about 75%, about 65% to about 85%, or about 75% to about 95% of the length of the fluid collection device 100.

The opening 106 in the fluid impermeable barrier 102 may exhibit a width that is measured transverse to the longitudinal axis of the fluid collection device 100 that may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid through the wicking material 115 and into the conduit 108. In some examples, the opening 106 may be vertically oriented (e.g., having a major axis parallel to the longitudinal axis of the device 100). In some examples (not shown), the opening 106 may be horizontally oriented (e.g., having a major axis perpendicular to the longitudinal axis of the device 100). In an example, the fluid impermeable barrier 102 may be attached to the individual, such as adhesively attached (e.g., with a hydrogel adhesive) to the individual. According to an example, a suitable adhesive is a hydrogel layer.

The fluid collection device 100 includes wicking material 115 disposed in the chamber 104. The wicking material 115 may cover at least a portion (e.g., all) of the opening 106. For example, at least a portion of the wicking material 115 may be exposed to an environment outside of the chamber 104 through the opening 106. The wicking material 115 may be configured to wick any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material. The wicking material 115 may also wick the fluid generally towards an interior of the chamber 104, as discussed in more detail below. The wicking material 115 may include one or more of a fluid permeable membrane 118 or a fluid permeable support 120.

The fluid permeable membrane 118 may include any material that may wick the fluid. For example, the fluid permeable membrane 118 may include fabric, such as a gauze (e.g., a silk, linen, or cotton gauze), another soft fabric, or another smooth fabric. The fluid permeable membrane 118 may include spun plastic fibers, such as a spun plastic mat or bed. Forming the fluid permeable membrane 118 from gauze, soft fabric, and/or smooth fabric may reduce chaffing caused by the fluid collection device 100.

The fluid collection device 100 may include the fluid permeable membrane 118 disposed in the chamber 104. The fluid permeable membrane 118 may cover at least a portion (e.g., all) of the opening 106. The fluid permeable membrane 118 may composed to wick any fluid away from the opening 106, thereby preventing the fluid from escaping the chamber 104.

The fluid collection device 100 may include the fluid permeable support 120 disposed in the chamber 104. The fluid permeable support 120 is configured to support the fluid permeable membrane 118 since the fluid permeable membrane 118 may be formed from a foldable, flimsy, or otherwise easily deformable material. For example, the fluid permeable support 120 may be positioned such that the fluid permeable membrane 118 is disposed between the fluid permeable support 120 and the fluid impermeable barrier 102. As such, the fluid permeable support 120 may support and maintain the position of the fluid permeable membrane 118. The fluid permeable support 120 may include any material that may wick the fluid, such as any of the fluid permeable membrane materials disclosed herein. For example, the fluid permeable membrane material(s) may be utilized in a more dense or rigid form than in the fluid permeable membrane 118 when used as the fluid permeable support 120. The fluid permeable support 120 may be formed from any fluid permeable material that is less deformable than the fluid permeable membrane 118, such as any of the materials disclosed herein for the fluid permeable membrane 118, in a more dense or rigid form. For example, the fluid permeable support 120 may include a porous polymer (e.g., nylon, polyester, polyurethane, polyethylene, polypropylene, etc.) structure or an open cell foam or spun plastic fibers. In some examples, the fluid permeable support 120 may be formed from a natural material, such as cotton, wool, silk, or combinations thereof. In such examples, the material may have a coating to prevent or limit absorption of fluid into the material, such as a water repellent coating. In some examples, the fluid permeable support 120 may be formed from fabric, felt, gauze, or combinations thereof. In some examples, the fluid permeable support 120 may be omitted from the fluid collection device 100. In some examples, the fluid permeable membrane 118 may be optional. For example, the wicking material 115 may include only the fluid permeable support 120.

The fluid permeable support 120 may have a greater ability to wick fluids than the fluid permeable membrane 118, such as to move the fluid inwardly from the outer surface of the fluid collection device 100. In some examples, the wicking ability of the fluid permeable support 120 and the fluid permeable membrane 118 may be substantially the same.

The fluid permeable membrane 118 and the fluid permeable support 120 may at least substantially completely fill the portions of the chamber 104 that are not occupied by the conduit 108. In another example, the fluid permeable membrane 118 and the fluid permeable support 120 may not substantially completely fill the portions of the chamber 104 that are not occupied by the conduit 108. In such an example, the fluid collection device 100 includes the reservoir (FIG. 1C) in the chamber 104.

The fluid collection device 100 includes the conduit 108, which extends into the chamber 104. The fluid collection device 100 may be operably coupled to a vacuum source. Accordingly, fluids may be removed from the chamber 104 via the conduit 108. The conduit 108 may extend into the chamber 104 to any point therein. For example, the conduit 108 may be inserted into the chamber at the first end region 125 of the fluid collection device 100 and extend therethrough to the second end region 127 of the fluid collection device 100.

The fluid impermeable barrier 102, the fluid permeable membrane 118 and the fluid permeable support 120 may be configured to have the conduit 108 at least partially disposed in the chamber 104. For example, at least one of the fluid permeable membrane 118 and the fluid permeable support 120 may be configured to form a space that accommodates the conduit 108. The fluid impermeable barrier 102 may define an aperture sized to receive the conduit 108. The at least one conduit 108 may be disposed in the chamber 104 via the aperture. The apertures may be configured to form an at least substantially fluid tight seal against the conduit 108 or the at least one tube thereby substantially preventing the fluid(s) from escaping the chamber 104. The fluid collected in the fluid collection device 100 may be removed from the chamber 104 via the conduit 108.

The fluid collection devices 100 disclosed herein include antimicrobial material 130 therein. The antimicrobial material 130 may be carried by one or more of the fluid impermeable barrier 102 or the wicking material 115. For example, antimicrobial material 130 may be disposed on, in, or incorporated into any of the fluid impermeable barrier 102, fluid permeable membrane 118, fluid permeable support 120, or the conduit 108, in one or more locations thereof.

The antimicrobial material may include one or more of an antibiotic material or an antifungal material. The antimicrobial material may include one or more of an antimicrobial organic compound, an inorganic compound, a polymeric biocidal or fungicidal material, metals, or any other material which reduces or prevents bacterial or fungal cell growth. Exemplary antimicrobial organic compounds may include organic acid salts, halogenated (e.g., chlorinated) polymers, nitrofurazone, organosilanes, or the like. Antimicrobial metals may include one or more of silver, titanium, cobalt, nickel, copper, brass, bronze, gold, zinc, zirconium, molybdenum, or tin. The antimicrobial material may include an oxide (e.g., zinc oxide), halide (e.g., chloride, bromide, iodide, etc.), sulfate, or another salt of any of the foregoing metals. The antimicrobial polymeric biocide may include polyhexamethylene guanidine hydrochloride (PHMGH), low density polyethylene (LDPE), nylon-3 polymers, polyethylene glycol(s), polymers containing phosphonium salts, ammonium salts, phenol groups, halogen-containing salts, or the like. Example antimicrobial inorganic materials may include sodium bicarbonate or the like As shown in FIGS. 1A and 1B, the antimicrobial material 130 may be incorporated into the wicking material 115, such as in the fluid permeable membrane 118. In some examples, the antimicrobial material 130 may be coated onto or impregnated in the fluid permeable membrane 118, the fluid permeable support 120, the fluid impermeable barrier 102, or the conduit 108. For example, one or both of the fluid permeable membrane 118 or fluid permeable support 120 may include fibers incorporating the antimicrobial material 130. In some examples, natural or synthetic fibers or a foam of the wicking material 115 may be coated with the antimicrobial material 130.

The antimicrobial material 130 may be woven into the wicking material 115, such as in the fluid permeable membrane 118 or the fluid permeable support 120. The antimicrobial material 130 may be disposed in or on threads in the fluid permeable membrane 118. For example, particles of silver, copper, aluminum, or another antimicrobial material may be disposed in the fibers or threads. In some examples, the antimicrobial fibers or threads may be present as less than 50% of the fibers or threads in the fluid permeable membrane 118, such as 1% to 50%, 1% to 10%, 5% to 25%, or 25% to 50% of the fibers or threads in the fluid permeable membrane 118. In some examples, the antimicrobial fibers or threads may be present as more than 50% of the fibers or threads in the fluid permeable membrane 118, such as in all of the threads of the fluid permeable membrane 118. In some examples, the antimicrobial material 130 may be coated onto the fibers or threads.

In some examples, the fibers or threads may be at least partially made of biocidal polymeric material. In some examples, the fibers or threads may be, or include, a wire of any of the antimicrobial materials disclosed herein.

In some examples, the antimicrobial material may be incorporated into the fluid impermeable barrier 102. For example, a polymer fluid impermeable barrier may include antimicrobial particles dispersed therein, such as any of the metals, polymers, or other antimicrobial materials disclosed herein. In some examples, antimicrobial material(s) may be less than 50 weight percent (wt %) of the fluid impermeable barrier 102, such as 0.1 wt % to 50 wt %, 0.1 wt % to 5 wt %, 1 wt % to 10 wt %, 10 wt % to 25 wt %, 20 wt % to 40 wt %, 25 wt % to 50 wt %, less than 25 wt %, less than 10 wt %, or more than 0.1 wt % of the fluid impermeable barrier 102.

As explained in more detail below, the antimicrobial material may be disposed on one or more surfaces of the fluid impermeable barrier 102, the wicking material 115, or the conduit 108. For example, the antimicrobial material may be disposed on one or more portions of at least one of the inner surface or the outer surface of the fluid impermeable barrier 102. In some examples, the antimicrobial material may be disposed in the wicking material as a powder applied thereto or may be coated thereon.

The conduit 108 may be at least partially disposed in the chamber 104. The conduit 108 (e.g., a tube) includes an inlet 110 at a first end region and an outlet 112 at a second end region positioned downstream from the inlet 110. The conduit 108 fluidly couples an interior region of the chamber 104 with the fluid storage container (not shown) or the vacuum source (not shown). The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 108 may include silicon or latex. In some examples, the conduit 108 may include one or more portions that are resilient, such as to by having one or more of a diameter or wall thickness that allows the conduit to be flexible.

In the illustrated embodiment of FIG. 1B, the conduit 108 is at least partially disposed in the chamber 104. For example, the conduit 108 may extend into the fluid impermeable barrier 102 from the first end region (e.g., proximate to the outlet 112) and may extend to the second end region (e.g., opposite the first end region) to a point proximate to a reservoir therein such that the inlet 110 is in fluid communication with the reservoir. In some examples (not shown), the conduit 108 may enter the chamber 104 in the second end region and the inlet 110 of the conduit 108 may be disposed in the second end region (e.g., in the reservoir). The fluid collected in the fluid collection device 100 may be removed from the chamber 104 via the conduit 108.

In some examples, the antimicrobial material 130 may be disposed on an outermost surface of the fluid collection device, such as one or more portions expected to be in contact with a wearer when in use. In some examples, the fluid collection device may include a reservoir therein. FIG. 1C is a cross-sectional view of the fluid collection device of FIG. 1A taken along the plane A-A, according to an embodiment. FIG. 1C depicts the fluid collection device 100c. The fluid collection device 100c includes the fluid impermeable barrier 102, the wicking material 115, the conduit 108, and the antimicrobial material 130 disposed on an outer surface of the fluid impermeable barrier 102 and the outer surface of the wicking material 115.

The antimicrobial material 130 may be disposed on one or more portions of at least one of the outer surface or the inner surface of the fluid impermeable barrier 102. In some examples, the antimicrobial material 130 may be incorporated into, or carried on, a material disposed on an outer surface of the fluid impermeable barrier 102. The antimicrobial material 130 may be disposed on the fluid collection device 100c as a coating on one or more portions thereof. The coating may include a carrier having a layer of antimicrobial material thereon or incorporating antimicrobial particles therein, such as a polymer having metal particles or nitrofurazone therein. The thickness of the antimicrobial material (e.g., coating) may be less than 1 mm, such as 1 μm to 1 mm, 1 μm to 10 mm, 1 μm to 10 μm, 10 μm to 100 μm, 100 μm to 500 μm, less than 500 μm, or less than 100 μm.

By disposing the antimicrobial material on the outer surface(s) of the fluid collection device 100c, bacterial and/or fungal growth may be reduced or eliminated while the fluid collection device 100c is in use. Accordingly, infections and odors may be reduced or eliminated compared to conventional fluid collection devices, particularly at points where the fluid collection device comes into contact with the wearer. Disposing the antimicrobial material on the portions of the fluid collection device may also aid in preventing the transfer of bacterial or fungal infections from the device to the wearer or vice versa.

While shown as being disposed on substantially the entire outer surface of the fluid collection device 100c, in some examples the antimicrobial material may only be disposed on an upper surface (e.g., portions of the fluid collection device at and around the opening 106) of the fluid collection device. For example, the antimicrobial material 130 may only be disposed on portions of the outer surface expected to be in contact with the user.

In some examples, the antimicrobial material 130 may be incorporated into or carried on a compression material disposed on the outer surface of the fluid impermeable barrier 102. For example, a compression bandage may have antimicrobial materials incorporated therein, such as having embedded antimicrobial polymer or metal particles or antimicrobial fibers. The compression bandage may have silver, copper, or aluminum particles or fibers therein. The compression bandage may be used to form an antimicrobial coating over one or more portions (e.g., cover the entire outer surface) of the fluid collection device 100c or may be utilized as the fluid permeable membrane 118. Suitable compression bandages may include natural and/or synthetic fibers therein, such as cotton, spandex, lycra, etc.

The antimicrobial material 130 may be located on one or more portions of the interior surface of the fluid impermeable barrier 102. By disposing the antimicrobial material on the interior surface of the fluid impermeable barrier 102, odors and microbial growth in the fluids collected therein may be reduced or eliminated.

In some examples, the antimicrobial material may be coated on or incorporated into the conduit 108. For example, a polymer conduit may include antimicrobial particles dispersed therein, such as any of the metals, polymers, or other antimicrobial materials disclosed herein. In some examples, antimicrobial material(s) may be less than 50 weight percent (wt %) of the conduit, such as 0.1 wt % to 50 wt %, 0.1 wt % to 5 wt %, 1 wt % to 10 wt %, 10 wt % to 25 wt %, 20 wt % to 40 wt %, 25 wt % to 50 wt %, less than 25 wt %, less than 10 wt %, or more than 0.1 wt % of the conduit. In some examples, the antimicrobial material may be coated onto one or more of an outer surface or an inner surface of the conduit 108. In such examples, bacterial or fungal growth may be limited or eliminated on or in the conduit 108.

As shown in FIG. 1C, the end of the conduit 108 may extend to the fluid permeable membrane 118 and/or fluid permeable support 120, such as into a reservoir 122. For example, the inlet 110 may extend into or be positioned in the reservoir 122. As shown, the reservoir 122 is a substantially unoccupied portion of the chamber 104. The reservoir may be defined between the fluid impermeable barrier 102 and one or both of the fluid permeable membrane 118 and the fluid permeable support 120. The fluid(s) that are in the chamber 104 may flow through the fluid permeable membrane 118 and/or fluid permeable support 120 to the reservoir 122. The fluid impermeable barrier 102 may retain the fluid(s) in the reservoir 122. While depicted in the second end region 127, the reservoir 122 may be located in any portion of the chamber 104 such as the first end region 125. In an example, the fluid impermeable barrier 102 may be air permeable and liquid impermeable. In such an example, the fluid impermeable barrier 102 may be formed of a hydrophobic material that defines a plurality of pores that are air permeable but not liquid permeable. In an example, at least one or more portions of at least an outer surface of the fluid impermeable barrier 102 may be formed from a soft and/or smooth material, thereby reducing chaffing.

In some examples, the inlet 110 may not extend into the reservoir 122. In such examples, the inlet 110 may be disposed within the wicking material 115 (fluid permeable membrane 118 and/or fluid permeable support 120) or at a terminal end thereof. For example, an end of the conduit 108 may be coextensive with or recessed within the fluid permeable membrane 118 and/or fluid permeable support 120.

In another example, the fluid permeable membrane 118 and the fluid permeable support 120 may not substantially completely fill the portions of the chamber 104 that are not occupied by the conduit 108. In such an example, the fluid collection device 100 includes the reservoir 122 disposed in the chamber 104. The reservoir 122 is a substantially unoccupied portion of the chamber 104. The reservoir may be defined between the fluid impermeable barrier 102 and one or both of the fluid permeable membrane 118 and the fluid permeable support. The fluid(s) that are in the chamber 104 may flow through the fluid permeable membrane 118 and/or fluid permeable support 120 to the reservoir 122. For example, the reservoir 122 may be located in a portion of the fluid collection device expected to be positioned in a gravimetrically low point of the fluid collection device when worn by a user. The reservoir 122 may store at least some of the fluid(s) therein.

In some examples, the fluid collection device 100 may include multiple reservoirs, such as a first reservoir that is located at the portion of the chamber 104 closest to the inlet 110 (e.g., second end region 127) and a second reservoir that is located at the portion of the of the chamber 104 that is closest to the outlet 112 (e.g., first end region 125). In another example, the fluid permeable support 120 is spaced from at least a portion of the conduit 108 and the reservoir 122 may be the space between the fluid permeable support 120 and the conduit 108.

The fluid impermeable barrier 102, the fluid permeable membrane 118 and the fluid permeable support 120 may be configured to have the conduit 108 at least partially disposed in the chamber 104. For example, at least one of the fluid permeable membrane 118 and the fluid permeable support 120 may be configured to form a space that accommodates the conduit 108. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 thereby substantially preventing the fluid(s) from escaping the chamber 104. The fluid collected in the fluid collection device 100 may be removed from the interior region of the chamber 104 via the conduit 108. As shown in FIG. 1C, the end of the conduit 108 may extend beyond the fluid permeable membrane 118 and/or fluid permeable support 120, such as into the reservoir 122. In some examples, the inlet 110 may not extend into the reservoir 122. In such examples, the inlet 110 may be disposed within the wicking material (fluid permeable membrane 118 and/or fluid permeable support 120) or a terminal end thereof. For example, an end of the conduit 108 may be coextensive with or recessed within the fluid permeable membrane 118 and/or fluid permeable support 120.

Locating the inlet 110 at or near a location expected to be the gravimetrically low point of the chamber 104 when worn by a user enables the conduit 108 to receive more of the fluid(s) than if inlet 110 was located elsewhere and reduce the likelihood of pooling (e.g., pooling of the fluid(s) may cause microbe growth and foul odors). For instance, the fluid(s) in the fluid permeable membrane 118 and the fluid permeable support 120 may flow in any direction due to capillary forces. However, the fluid(s) may exhibit a preference to flow in the direction of gravity, especially when at least a portion of the fluid permeable membrane 118 and/or the fluid permeable support 120 is saturated with the fluid(s). Accordingly, one or more of the inlet 110 or the reservoir 122 may be located in the second end region 127.

In an example, the conduit 108 is configured to be at least insertable into the chamber 104. In such an example, the conduit 108 may include one or more markers (not shown) on an exterior thereof that are configure to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another example, the conduit 108 may include one or more markings thereon that are configured to facilitate correct rotation of the conduit 108 relative to the chamber 104. In an example, the one or more markings may include a line, a dot, a sticker, or any other suitable marking.

The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of an alignment guide or an orientation indicator, such as a stripe or hashes. Such markings may be positioned to align the device 100 to one or more anatomical features such as a pubic bone, etc.

Other embodiments of fluid impermeable barriers, fluid permeable membranes, fluid permeable supports, chambers, and their shapes and configurations are disclosed in U.S. patent application Ser. No. 15/612,325 filed on Jun. 2, 2017; U.S. Pat. No. 10,390,989 filed on Sep. 8, 2016; and U.S. Pat. No. 10,226,376 filed on Jun. 1, 2017, the disclosure of each of which is incorporated herein, in its entirety, by this reference.

In an example, one or more components (e.g., fluid impermeable barrier 102, conduit 108, the wicking material 115, etc.) of the fluid collection device 100 may include an odor blocking or absorbing material such as a cyclodextrine-containing material or a thermoplastic elastomer (TPE) polymer.

As described in more detail below, the conduit 108 is configured to be coupled to and at least partially extend between one or more of the fluid storage container (not shown) and the vacuum source (not shown). In an example, the conduit 108 is configured to be directly connected to the vacuum source (not shown). In such an example, the conduit 108 may extend from the fluid impermeable barrier 102 by at least one foot, at least two feet, at least three feet, or at least six feet. In another example, the conduit 108 may be indirectly connected to at least one of the fluid storage container (not shown) and the vacuum source (not shown). In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 may fluidly couple (e.g., directly or indirectly) the vacuum source (not shown) to the chamber 104 (e.g., the reservoir 122), such as via one or more connectors thereon. In an example, the inlet 110 and/or the outlet 112 may form a male connector. In another example, the inlet 110 and/or the outlet 112 may form a female connector. In an example, the inlet 110 and/or the outlet 112 may include ribs that are configured to facilitate secure couplings. In an example, the inlet 110 and/or the outlet 112 may form a tapered shape. In an example, the inlet 110 and/or the outlet 112 may include a rigid or flexible material.

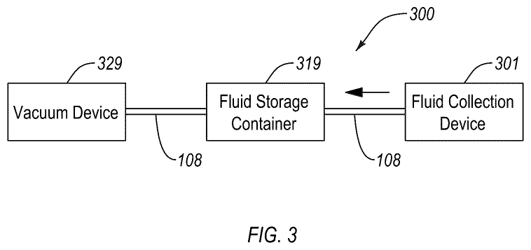

As the vacuum source (FIG. 3) applies a vacuum/suction in the conduit 108, the fluid(s) in the chamber 104 (e.g., at the second end region such as in the reservoir 122) are drawn into the inlet 110 and out of the fluid collection device 100 via the conduit 108. In some examples, the conduit may be frosted or opaque (e.g., black) to obscure visibility of the fluid(s) therein.

The fluid collection devices shown in FIGS. 1A-1C are examples of female fluid collection devices that are configured to collect fluid(s) from females. However, the fluid collection devices, systems, and methods disclosed herein may include male fluid collection devices shaped, sized, and otherwise configured to collect fluid(s) from males.

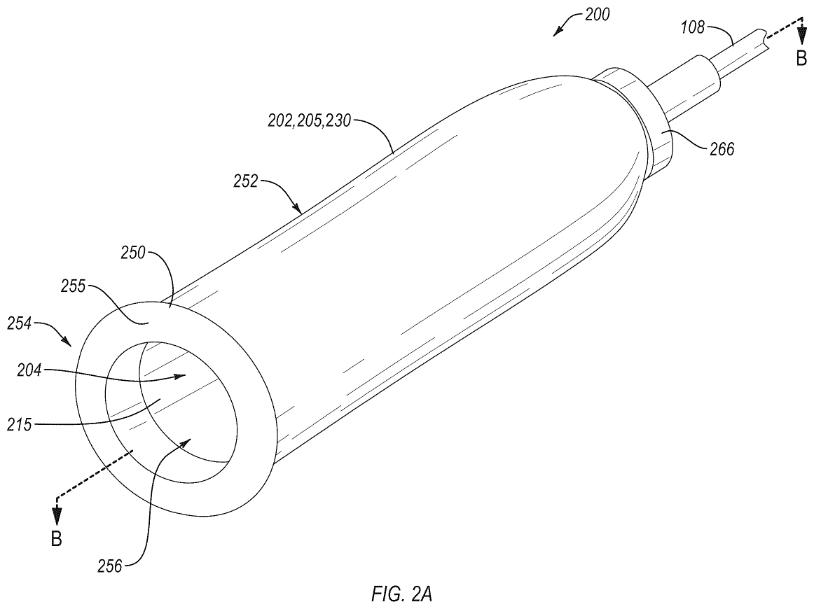

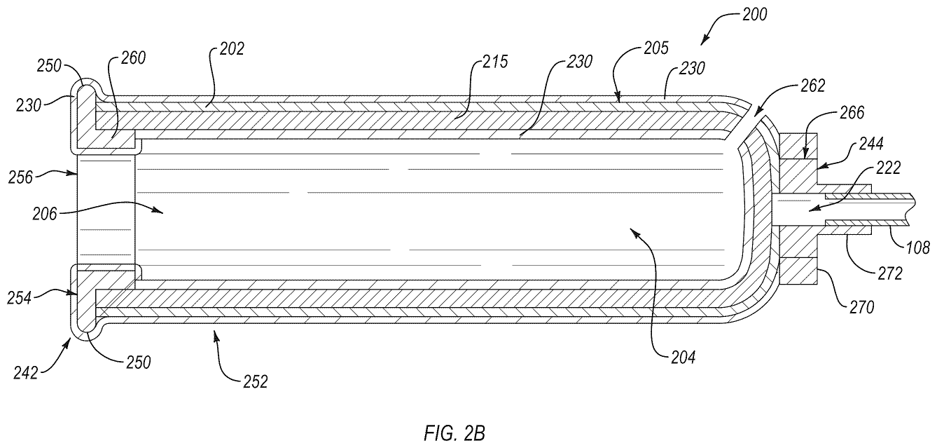

FIG. 2A is an isometric view of a fluid collection device 200, according to an embodiment. FIG. 2B is a cross-sectional view of a fluid collection device 200 of FIG. 2A along the plane B-B, according to an embodiment. Referring to FIGS. 2A and 2B, the fluid collection device 200 includes a receptacle 250 and a sheath 252 (e.g., cup shaped container). The antimicrobial material 230 may be disposed on one or more surfaces of at least one of the receptacle 250 (including the wicking material disposed therein), the sheath 252, the cap 266, or the conduit 108. The antimicrobial material 230 may be similar or identical to the antimicrobial material 130 disclosed herein, in one or more aspects. For example, the antimicrobial material 230 may include one or more of an antimicrobial organic compound, an inorganic compound, a polymeric biocidal or fungicidal material, metals, or any other material which reduces or prevents bacterial or fungal cell growth as disclosed herein.

The receptacle 250 is sized, shaped, and made of a material to be coupled to skin that surrounds the male urethra and have the male urethra positioned therethrough. For example, the receptacle 250 may include an annular base 254 that defines an opening 256 in the receptacle 250. The annular base 254 is sized and shaped to be positioned around the male urethra (e.g., positioned around the penis) and the opening 256 may be configured to have the male urethra positioned therethrough. The annular base 254 may also be sized, shaped, made of a material, or otherwise configured to be coupled (e.g., adhesively attached, such as with a hydrogel adhesive) to around the male urethra (e.g., around the penis). In an example, the annular base 254 may exhibit the general shape of the skin surface that the annular base 254 is selected to be coupled with and/or may be flexible thereby allowing the annular base 254 to conform to any shape of the skin surface. The receptacle 250 also defines a hollowed region that is configured to receive (e.g., seal against) the sheath 252. For example, the receptacle 250 may include a flange 260 that extends upwardly from the annular base 254. The flange 260 may be tall enough to prevent the sheath 252 from being accidentally removed from the receptacle 250 (e.g., at least 0.5 cm tall, 1 cm tall, at least 2 cm tall, or at least 5 cm tall). While depicted as being disposed within the sheath 252, in some examples the flange 260 may be disposed on the outer surface of the sheath 252.

The receptacle 250 may be formed of any of the materials disclosed herein for the fluid impermeable barrier 102, such as a silicone polymer.

In some examples, the antimicrobial material 230 may be disposed on one or more of the outer surface or the inner surface of the receptacle 250. The antimicrobial material 230 may be present as a coating, such as a coating of any of the antimicrobial materials disclosed herein. For example, the antimicrobial coating may be disposed on the annular base 254 or in the opening 256 where the fluid collection device 200 contacts the skin of a wearer, such as to prevent or limit growth or transmission of bacteria and fungi from the fluids to the wearer. The coating may include a carrier having antimicrobial particles therein, such as a polymer having metal particles or nitrofurazone therein. In some examples, the antimicrobial material 230 may be incorporated into the material of the receptacle 250, such as antimicrobial particles (e.g., metals) or polymers embedded in the receptacle material as disclosed herein with respect to the fluid impermeable barrier 102 of the fluid collection device 100. For example, the receptacle 250 may be formed of silicone having silver, copper, or aluminum impregnated therein.

The sheath 252 includes (e.g., may be formed from) a fluid impermeable barrier 202 that is sized and shaped to fit into or around opening 256 (e.g., the hollowed region) and/or flange 260 of the receptacle 250. The fluid impermeable barrier 202 may be disposed on wicking material 215. The wicking material 215 may be similar or identical to the wicking material 115 disclosed herein, in one or more aspects. For example, the wicking material 215 may include one or more of the fluid permeable membrane or the fluid permeable support as disclosed herein with respect to the wicking material 115. The fluid impermeable barrier 202 of the sheath 252 may be shaped to contain, and allow for, different sizes and states of male penises. For example, the sheath 252 may be generally cup-shaped or tubular with a substantially closed end. For example, the sheath 252 may be substantially cylindrical with a closed distal end. The cylindrical sheath 252 may be substantially cylindrical in that the cylinder may be deformable and at least partially collapsible/expandable based on the size and state of the penis of the wearer. Accordingly, the penis of a wearer may be inserted into and contained within the sheath 252 whether in a flaccid or erect state.

The fluid impermeable barrier 202 may be similar or identical to the fluid impermeable barrier 102, in one or more aspects such as material composition, thickness, fluid retention, etc. The fluid impermeable barrier 202 at least partially defines a chamber 204 therein. The fluid impermeable barrier 202 may also define an opening 206 extending through the fluid impermeable barrier 202 to the chamber 204.

In some examples, the antimicrobial material 230 may be disposed on one or more of the outer surface or the inner surface of the fluid impermeable barrier 202. The antimicrobial material 230 may be present as a coating, such as any of the coatings disclosed herein. In some examples, the antimicrobial material 230 may be incorporated into the fluid impermeable barrier, such as disclosed herein with respect to the fluid collection device 100.

The fluid impermeable barrier 202 may also include at least one aperture 262 that allows the chamber 204 to remain substantially at atmospheric pressure even when suction or a vacuum is applied in the chamber 204. The aperture 262 may be located on any portion of the sheath 252, such as a portion not intended to serve as a reservoir for collected fluids. In some examples (not shown), the aperture 262 may extend through the cap 266 or be disposed beneath the cap 266. In some examples, the fluid collection device 200 may not include the aperture 262, such as when a more complete seal as desired for the chamber 204.

The fluid impermeable barrier 202 may have the wicking material 215 in an inner surface thereof to form a fluid impermeable barrier separating the interior (e.g., chamber 204) of the fluid collection device 200 from the external environment. One or more of the fluid permeable membrane or the fluid permeable support may extend around at least a portion of the inner surface of the fluid impermeable barrier 202. In some examples, the fluid permeable membrane or the fluid permeable support may be omitted from the wicking material 215. One or more portions of the fluid impermeable barrier 202 may be adhered to the outer surface of the wicking material 215 (e.g., the fluid permeable membrane or the fluid permeable support). Put another way, the fluid permeable support may be disposed on an interior surface of the fluid impermeable barrier 202. The fluid permeable membrane may be disposed on an interior surface of the fluid permeable support, such as disposed concentrically within the fluid permeable support. In such examples, the fluid permeable membrane may contact the skin of the wearer (e.g., penis) when in use. In some examples (not shown), the fluid permeable membrane may be located at the outer surface of the wicking material 215, wherein the fluid impermeable barrier 202 is adhered to the outer surface of the fluid permeable membrane.

The antimicrobial material 230 may be located on or incorporated into the wicking material 215 as disclosed with respect to the fluid collection device 100. For example, the antimicrobial material may include fibers or threads bearing antimicrobial material (e.g., silver or copper) therein. As shown in FIG. 2B, the antimicrobial material 230 may be a coating applied to the innermost surface of the wicking material 215.

To facilitate fluid collection and provide comfort, the sheath 252 may be flexible, relatively soft, and have a relatively smooth outer surface, thereby allowing the sheath 252 to correspond to the shape of a penis. For example, the sheath 252 may at least partially collapse when the penis is flaccid and at least partially expand and bend to the shape of the penis as the penis becomes erect.

The receptacle 250 may be more rigid than the sheath 252. For example, the receptacle 250 may be formed from a flexible polymer that is at least one of thicker than the entire sheath 252 or exhibits a Young's modulus that is greater than sheath 252. As such, the receptacle 250 may provide some structure at or near the proximal end 242 of the fluid collection device 200. The higher rigidity of the receptacle 250 may cause the receptacle 250 to remain open, thereby facilitating insertion of the urethra (e.g., penis) into the fluid collection device 200. The relatively high rigidity of the receptacle 250 enables the receptacle 250 to act as an attachment point to a wearer, wicking material 215, and/or the fluid impermeable barrier 202. In some examples, the receptacle 250 may define a recess, include threads, or include any other attachment substrate for attaching the components of the fluid collection device 200, such as the wicking material 215 or the fluid impermeable barrier 202.

In some examples, the fluid collection device 200 includes a cap 266 at a distal end region 244. The cap 266 defines a in interior channel through which the fluids may be removed from the fluid collection device 200. The interior channel is in fluid communication with the chamber 204. The cap 266 may be disposed over at least a portion of the distal end region 244 of one or more of the fluid impermeable barrier 202 or the wicking material 215. The cap 266 may be made of a polymer, rubber, or other fluid impermeable material. The cap 266 may be attached to one or more of the fluid impermeable barrier 202, the wicking material 215, or the conduit 108. The cap 266 may have a laterally extending flange 270 and a longitudinally extending flange 272. The laterally extending flange 270 may cover at least a portion of the distal end region 244 of the fluid collection device 200. The laterally extending flange 270 may extend laterally along the outer surface of the end of the sheath 252. The longitudinally extending flange 272 may extend a distance from the sheath 252. The longitudinally extending flange 272 is sized and configured to receive and fluidly seal against the conduit 108, such as within the interior channel. The conduit 108 may extend a distance within the cap 266, such as to the wicking material 215, through the wicking material 215, or to a point set-off from the wicking material 215. In the latter example, as depicted in FIG. 2B, the interior channel of the cap 266 may define a reservoir 222 therein. The reservoir 222 is an unoccupied portion of the device such as in the cap 266 and is void of other material. In some examples, the reservoir 222 is defined at least partially by the wicking material 215 and the cap 266. During use, the fluids that are in the chamber 204 may flow through the wicking material 215 to the reservoir 222. The reservoir 222 may store at least some of the fluids therein and/or position the fluids for removal by the conduit 108. In some examples, at least a portion of the wicking material 215 may extend continuously between at least a portion of the opening of the interior channel and chamber 204 to wick any fluid from the opening directly to the reservoir 222. In some examples, the reservoir may be defined between the interior surface of the fluid impermeable barrier and the penis of a wearer, such as an unoccupied portion of the chamber 204.

In some examples (not shown), the fluid impermeable barrier 202 may be disposed on the cap 266, such as enclosing the cap 266 within the chamber 204.

In some examples, the sheath 252 may include at least a portion of the conduit 108 therein, such as at least partially disposed in the chamber 204. For example, the conduit 108 may extend from the sheath 252 to a region at least proximate to the opening 256. For example, the inlet of the conduit may be positioned adjacent to the annular base 254. The region proximate to the opening 256 may be disposed near or on the skin around the male urethra (e.g., around the penis). Accordingly, when a patient lays on their back, fluid (e.g., urine) may aggregate near the opening 206 against the skin of the wearer. The fluid may be removed from the chamber 204 via the conduit 108. The conduit 108 includes the inlet positioned within the fluid collection device and an outlet configured to be fluidly coupled to a vacuum source. The antimicrobial material 230 may be disposed on the outer surface, inner surface, or incorporated into, the material of the conduit 108.

The receptacle 250, the sheath 252, the cap 266, and the conduit 108 may be attached together using any suitable method. For example, at least two of the receptacle 250, the sheath 252, the cap 266, or the conduit 108 may be attached together using at least one of an interference fit, an adhesive, stitching, welding (e.g., ultrasonic welding), tape, any other suitable method, or combinations thereof. The antimicrobial material may be located on one or more portions of the receptacle, the sheath, the cap, or the conduit.

In some examples, the vacuum source (not shown) may be remotely located from the sheath 252. In such examples, the conduit 108 may extend out of and away from the sheath 252 to the vacuum source or a fluid storage container. For example, the inlet 110 of the conduit may be used to remove fluid from the chamber 204 via vacuum when an outlet (not shown) of the conduit 108 is fluidly coupled to the vacuum source or fluid storage container operably coupled to the vacuum source.

In some examples (not shown), the fluid collection device 200 does not include the cap. In such examples, the outlet of the conduit 108 may be fluidly coupled to a fluid storage container (not shown). In such examples, the fluid impermeable barrier 202 may include at least one aperture sized and shaped to receive and seal against the conduit 108, such as within the chamber 204. Accordingly, the chamber 204 may be fluidly coupled to the vacuum source via the conduit 108. As the vacuum source applies a vacuum/suction, the fluid in the chamber 204 may be removed through the conduit 108. In some examples, the fluid may be pumped through the vacuum source (not shown) into a section of the conduit 108 fluidly coupled to a fluid storage container (not shown) into which the fluid may be deposited.

In examples, portions of the chamber 204 may be substantially empty due to the varying sizes and rigidity of the male penis. However, in some examples (not shown), the outermost regions of the chamber 204 (e.g., periphery of the interior regions of the sheath 252) may include wicking material 215 in a position to blunt a stream of urine from the male urethra thereby limiting splashing and/or to direct the fluids to a selected region of the chamber 204. Since the chamber 204 is substantially empty (e.g., substantially all of the chamber 204 forms a reservoir), the fluids are likely to pool at a gravimetrically low point of the chamber 204. Depending on the orientation of the wearer, the gravimetrically low point of the chamber 204 may be at an intersection of the skin of a wearer and the fluid collection device 200 (proximate to opening 256), a corner formed in the sheath 252, or another suitable location (e.g., proximate to a region opposite the opening 256). The inlet of the conduit 108 may be positioned to be adjacent or proximate to the gravimetrically low point of the chamber 204.

During operation, a male using the fluid collection device 200 may discharge fluids (e.g., urine) into the chamber 204. The fluids may pool or otherwise be collected in the chamber 204, such as against the skin of the user (e.g., wearer). At least some of the fluids may enter the interior of the conduit 108 via the inlet of the conduit. The fluid may be drawn out of the fluid collection device 200 via the vacuum/suction provided by the vacuum source. When present and during operation, the aperture 262 may substantially maintain the pressure in the chamber 204 at atmospheric pressure even though fluid is introduced into and subsequently removed from the chamber 204.

The fluid collection devices disclosed herein are used in fluid collection systems to collect and remove fluids from a wearer of the device. As the vacuum source (FIG. 3) applies a vacuum/suction in the conduit 108, the fluid(s) in the chamber 104 or 204 (e.g., at the second end region such as in the reservoir 222) may be drawn into the inlet and out of the fluid collection device 100 or 200 via the conduit 108.

FIG. 3 is a block diagram of a system 300 for fluid collection, according to an embodiment. The system 300 includes a fluid collection device 301, a fluid storage container 319, and a vacuum source 329. The fluid collection device 301, the fluid storage container 319, and the vacuum source 329 may be fluidly coupled to each other via one or more conduits 108. For example, fluid collection device 301 may be operably coupled to one or more of the fluid storage container 319 or the (portable) vacuum source 329 via the conduits 108.

The fluid collection device 301 may be similar or identical to any of the fluid collection devices disclosed herein, such as a male or female fluid collection device. For example, the fluid collection device 301 may include a fluid impermeable barrier having an outer surface and an inner surface, where the inner surface at least partially defines a chamber within the fluid collection device 301. The fluid impermeable barrier also defines an opening configured to be positioned adjacent to a female urethra or have a male urethra positioned therethrough. The fluid impermeable barrier may be similar or identical to the any of the fluid impermeable barriers disclosed herein. The fluid collection device 301 includes a wicking material disposed within the fluid impermeable barrier, such as any of the wicking materials disclosed herein. The fluid collection device 301 may include a conduit including an inlet and an outlet as disclosed herein. The outlet may be fluidly coupled to the fluid storage container and the inlet may be positioned in a portion of the chamber selected to be at a gravimetrically low point of the fluid collection device when worn by a user. The fluid collection device 301 may an antimicrobial material carried by one or more of the fluid impermeable barrier, the wicking material, or the conduit as disclosed herein. The antimicrobial material may be an antibiotic or antifungal material, such as any of those disclosed herein. The antimicrobial material may inhibit or prevent the growth and transmission of bacteria, fungi, or other unwanted microbes when utilizing the fluid collection devices and systems disclosed herein.

Fluid (e.g., urine or other bodily fluids) collected in the fluid collection device 301 may be removed from the fluid collection device 301 via the conduit 108 which protrudes into an interior region of the fluid collection device 301. For example, a first open end of the conduit 108 may extend into the fluid collection device 301 to a reservoir therein. The second open end of the conduit 108 may extend into the fluid collection device 301 or the vacuum source 329. The suction force may be introduced into the interior region of the fluid collection device 301 via the first open end of the conduit 108 responsive to a suction (e.g., vacuum) force applied at the second end of the conduit 108. The suction force may be applied to the second open end of the conduit 108 by the vacuum source 329 either directly or indirectly.

The suction force may be applied indirectly via the fluid storage container 319. For example, the second open end (e.g., outlet) of the conduit 108 may be disposed within the fluid storage container 319 and an additional conduit 108 may extend from the fluid storage container 319 to the vacuum source 329. Accordingly, the vacuum source 329 may indirectly apply suction to the fluid collection device 301 via the fluid storage container 319. In such examples, the vacuum source 329 may provide a vacuum/suction through the fluid storage container to the fluid collection device to provide suction in the chamber of the fluid collection device. Accordingly, a vacuum (e.g., suction) may be drawn through fluid collection device 301 via the fluid storage container 319. As the fluid is drained from the chamber, the fluid may travel through the first section of conduit to the fluid storage container where it may be retained. Fluid, such as urine, may be drained from the fluid collection device 301 using the vacuum source 329.

In some examples, the suction force may be applied directly via the vacuum source 329. For example, the second open end of the conduit 108 may be disposed within the vacuum source 329. An additional conduit 108 may extend from the vacuum source 329 to a point outside of the fluid collection device 301, such as to the fluid storage container 319. In such examples, the vacuum source 329 may be disposed between the fluid collection device 301 and the fluid storage container 319. In examples, the fluid storage container 319 may include a bag (e.g., drainage bag), a bottle or cup (e.g., collection jar), or any other enclosed container for storing bodily fluids such as urine.

The vacuum source 329 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 329 may provide a vacuum or suction to remove fluid from the fluid collection device 301. In examples, the vacuum source 329 may be powered by one or more of a power cord (e.g., connected to a power socket), one or more batteries, or even manual power (e.g., a hand operated vacuum pump). The vacuum sources 329 disclosed herein may include one or more of a switch, a button, a plug, a remote, or any other device suitable to activate the vacuum source 329.

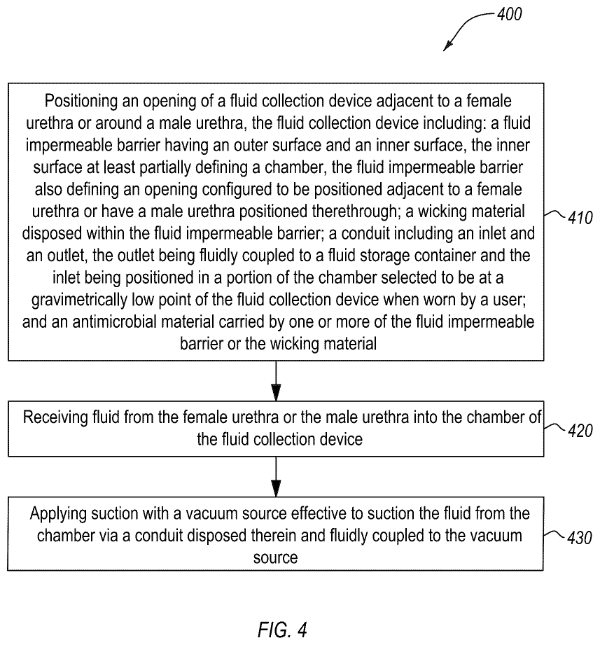

FIG. 4 is a flow diagram of a method 400 to use any of the fluid collection devices and/or fluid collection systems disclosed herein, according to an example. The method 400 may include act 410, which recites “positioning an opening of a fluid collection device adjacent to a female urethra or around a male urethra, the fluid collection device including a fluid impermeable barrier having an outer surface and an inner surface, the inner surface at least partially defining a chamber, the fluid impermeable barrier also defining an opening configured to be positioned adjacent to a female urethra or have a male urethra positioned therethrough; a wicking material disposed within the fluid impermeable barrier; a conduit including an inlet and an outlet, the outlet being fluidly coupled to a fluid storage container and the inlet being positioned in a portion of the chamber selected to be at a gravimetrically low point of the fluid collection device when worn by a user; and an antimicrobial material carried by one or more of the fluid impermeable barrier or the wicking material.” Act 410 may be followed by act 420, which recites “receiving fluid from the female urethra or the male urethra into a chamber of the fluid collection device.” Act 420 may be followed by act 430, which recites “applying suction with a vacuum source effective to suction the fluid from the chamber via a conduit disposed therein and fluidly coupled to the vacuum source.”

Acts 410, 420, 430 of the method 400 are for illustrative purposes. For example, the act 410, 420, 430 of the method 400 may be performed in different orders, split into multiple acts, modified, supplemented, or combined. In an example, one or more of the acts 410, 420, 430 of the method 400 may be omitted from the method 400. Any of the acts 410, 420, or 430 may include using any of the fluid collection devices or systems disclosed herein.

Act 410 recites “positioning an opening of a fluid collection device adjacent to a female urethra or around a male urethra, the fluid collection device including a fluid impermeable barrier having an outer surface and an inner surface, the inner surface at least partially defining a chamber, the fluid impermeable barrier also defining an opening configured to be positioned adjacent to a female urethra or have a male urethra positioned therethrough; a wicking material disposed within the fluid impermeable barrier; a conduit including an inlet and an outlet, the outlet being fluidly coupled to a fluid storage container and the inlet being positioned in a portion of the chamber selected to be at a gravimetrically low point of the fluid collection device when worn by a user; and an antimicrobial material carried by one or more of the fluid impermeable barrier or the wicking material.” The fluid collection device or components thereof may be similar or identical to any of the fluid collection devices (e.g., 100 or 200) disclosed herein, in one or more aspects.

In some examples, act 410 may include positioning the opening of a female fluid collection device such that the fluid permeable membrane of the female fluid collection device abuts or is positioned proximate to (e.g., over) the female urethra. In some examples, act 410 may include positioning a receptacle of a male fluid collection device around (e.g., over) the male urethra such that the male urethra is positioned in the receptacle. In such an example, act 410 may include positioning the sheath of the male fluid collection device in a hollowed region of the receptacle such that the male urethra is positioned through an opening of the sheath of the male fluid collection device and into the chamber of the male fluid collection device. In some examples, the act 410 may include positioning a penis within the fluid collection device, such as in the chamber thereof. In some examples, positioning an opening of a fluid collection device adjacent to a female urethra or around a male urethra may include positioning the opening over the female urethra, such as positioning a longitudinally extending opening of the fluid collection device over the female urethra.

Act 420 recites “receiving fluid from the female urethra or the male urethra into the chamber of the fluid collection device.” For example, act 420 may include wicking the fluid(s) away from the opening using a fluid permeable membrane and a fluid permeable support. In some examples, act 420 may include receiving the fluid(s) into the chamber of the sheath of the male fluid collection device. In either example, act 420 may include flowing the fluid towards a portion of the chamber that is fluidly coupled to an inlet of a conduit, which may be in fluid communication a vacuum source. For instance, act 420 may include flowing the fluid(s) to a substantially unoccupied portion of the chamber (e.g., a reservoir), to a gravimetrically low point of the chamber, etc. In some examples, receiving fluid(s) from the female urethra or the male urethra into a chamber of the fluid collection device may include wicking the fluid (e.g., urine) into the chamber via the fluid permeable membrane and fluid permeable support of the fluid collection device. For example, wicking the fluid into the chamber via the fluid permeable membrane and fluid permeable support may include wicking urine into a reservoir in the fluid collection device.

Act 430 recites, “applying suction with a vacuum source effective to suction the fluid(s) from the chamber via a conduit disposed therein and fluidly coupled to the vacuum source.” In some examples, applying suction with a vacuum source effective to suction the fluid(s) from the chamber via a conduit disposed therein and fluidly coupled to the vacuum source may include using any of the vacuum sources disclosed herein. In an example, act 430 may include activating the vacuum source (e.g., suction device) in fluid communication with the inlet of the conduit in the fluid collection device. In some examples, activating the vacuum source in fluid communication with the inlet of the conduit in the fluid collection device may include supplying power to the vacuum source by one or more of flipping an on/off switch, pressing a button, plugging the vacuum source into a power outlet, putting batteries into the vacuum source, etc. In some examples, the vacuum source may include a hand operated vacuum pump and applying suction with a vacuum source may include manually operating the hand operated vacuum pump effective to suction the fluid(s) from the chamber via the conduit disposed therein that is fluidly coupled to the vacuum source.

In some examples, applying suction with a vacuum source effective to suction the fluid(s) from the chamber via a conduit disposed therein and fluidly coupled to the vacuum source may be effective to remove at least some fluid (e.g., urine) from the chamber (e.g., interior region) of the fluid collection device. In some examples, applying suction with a vacuum source effective to suction the fluid(s) from the chamber via a conduit disposed therein and fluidly coupled to the vacuum source may be effective to transfer at least some of the fluid from the chamber of the fluid collection device to a fluid storage container (e.g., a bottle or bag). In some examples, applying suction with a vacuum source effective to suction the fluid(s) from the chamber may include removing fluid from one or more of a reservoir, fluid permeable support, or fluid permeable membrane of the fluid collection device.

In some examples, the vacuum source (e.g., suction device) may be disposed on or within the fluid collection device. In some examples, the vacuum source may be spaced from the fluid collection device.

In some examples, applying suction with a vacuum source effective to suction the fluid(s) from the chamber via a conduit disposed therein and fluidly coupled to the vacuum source may include detecting moisture in the chamber (e.g., via one or more moisture sensors) and responsive thereto, activating the vacuum source to provide suction in the chamber. The control of the vacuum source responsive to the signals indicating that moisture or a level thereof is present in the chamber may be automatic, such as via a controller (e.g., computer programmed to perform the operation), or may merely provide an indication that a level of moisture is present that may necessitate removal of fluid from the chamber of the fluid collection device. In the latter case, a user may receive the indication (e.g., from the controller) and activate the vacuum pump manually.

In an example, the method 400 may include collecting the fluid(s) that are removed from the fluid collection device, such as into a fluid storage container that is spaced from the fluid collection device and fluidly coupled to the conduit. The fluid storage container may include any of the fluid storage containers disclosed herein.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

1. A fluid collection device, comprising:
a fluid impermeable barrier having an outer surface and an inner surface, the inner surface at least partially defining a chamber, the fluid impermeable barrier also defining an opening extending therethrough, the opening is configured to be positioned adjacent to a female urethra or a male urethra;
a fluid permeable material disposed at least partially in the chamber and positioned to contact skin of a wearer;
an antimicrobial material carried by at least the fluid impermeable barrier, wherein the antimicrobial material is disposed on the outer surface of the fluid impermeable barrier that are is expected to be in contact with skin of a wearer when in use.

2. The fluid collection device of claim 1, wherein the antimicrobial material is carried by the fluid impermeable barrier and disposed only on the outer surface of the fluid impermeable barrier that is expected to be in contact with the skin of the wearer when the fluid collection device is in use.

3. The fluid collection device of claim 1, wherein the antimicrobial material is disposed on the outer surface of the fluid impermeable barrier that is positioned to be in contact with external skin of the wearer when the fluid collection device is in use.

4. The fluid collection device of claim 1, wherein the fluid permeable material includes a fluid permeable membrane disposed at least partially within the chamber and a fluid permeable support disposed at least partially within the chamber.

5. The fluid collection device of claim 4, wherein the fluid permeable material is positioned to contact skin of the wearer, with the fluid permeable membrane having an outer surface extending across the opening and the fluid permeable support positioned to support the fluid permeable membrane.

6. The fluid collection device of claim 5, wherein the antimicrobial material is carried by the fluid permeable material and disposed on the outer surface of the fluid permeable membrane that is expected to be positioned at least proximate to the female urethra and outside the female urethra when the fluid collection device is in use.

7. The fluid collection device of claim 6, wherein:
the antimicrobial material includes one or more of a polymeric biocide, silver, copper, or aluminum; and
one or both of the fluid permeable membrane or fluid permeable support include fibers incorporating the antimicrobial material.

8. The fluid collection device of claim 1, wherein the antimicrobial material includes one or more of an antibiotic material or an antifungal material.

9. The fluid collection device of claim 1, wherein the antimicrobial material includes one or more of an organic compound, a polymeric biocide, silver, copper, or aluminum.

10. The fluid collection device of claim 1, wherein the antimicrobial material is disposed on the inner surface.

11. The fluid collection device of claim 1, wherein the antimicrobial material is incorporated into a material of the fluid impermeable barrier.

12. The fluid collection device of claim 1, further comprising a conduit including an inlet and an outlet, the inlet being positioned within the fluid collection device and the outlet is configured to be fluidly coupled to a fluid storage container.

13. The fluid collection device of claim 12, wherein:
the fluid impermeable barrier and the fluid permeable material define a reservoir therebetween; and
the inlet is disposed in the reservoir.

14. The fluid collection device of claim 12, further comprising:
a fluid storage container configured to hold a fluid and in fluid communication with the chamber of the fluid impermeable barrier through the conduit; and
a vacuum source fluidly coupled to one or more of the fluid storage container or the fluid collection device via the conduit, the vacuum source configured to draw fluid from the fluid collection device via the conduit.

15. A method to collect fluid, the method comprising:
providing a fluid collection device including:
a fluid impermeable barrier having an outer surface and an inner surface, the inner surface at least partially defining a chamber, the fluid impermeable barrier also defining an opening extending therethrough;
a fluid permeable material disposed at least partially in the chamber and positioned to contact skin of a wearer; and
an antimicrobial material carried by at least the fluid impermeable barrier,
wherein the antimicrobial material is disposed on at least some of the outer surface of the fluid impermeable barrier;
positioning the opening of the fluid collection device adjacent to a female urethra with at least some of the outer surface of the fluid impermeable barrier having the antimicrobial material disposed thereon contacting skin of the wearer;
receiving fluid from the female urethra into the chamber of the fluid collection device; and
applying suction with a vacuum source effective to suction the fluid from the chamber via a conduit disposed therein and fluidly coupled to the vacuum source.

16. The method of claim 15, wherein:
the fluid permeable material includes a fluid permeable membrane disposed at least partially within the chamber and a fluid permeable support disposed at least partially within the chamber;
positioning the opening of the fluid collection device adjacent to a female urethra includes positioning the fluid permeable material to contact skin of the wearer, with the antimicrobial material being carried by the fluid permeable material and disposed on the outer surface of the fluid permeable membrane that is positioned at least proximate to the female urethra and outside the female urethra when the fluid collection device.

17. The method of claim 16, wherein:
the antimicrobial material includes one or more of a polymeric biocide, silver, copper, or aluminum; and
one or both of the fluid permeable membrane or fluid permeable support include fibers incorporating the antimicrobial material.

18. The method of claim 15, wherein the antimicrobial material includes one or more of an antibiotic material, an antifungal material, an organic compound, a polymeric biocide, silver, copper, or aluminum.

19. The method of claim 15, wherein the antimicrobial material is disposed on the inner surface.

20. The method of claim 15, wherein the antimicrobial material is incorporated into a material of the fluid impermeable barrier.
